(19) United States
(12) Patent Application Publication
(10) Pub. No.: US 2026/0354785 A1
(43) Pub. Date: Nov. 12, 2026

(54) DETECTING FAULTY CABLES, CABLE CONNECTIONS AND/OR CIRCUIT BOARDS DURING CT IMAGING SYSTEM ASSEMBLY BEFORE FULL SYSTEM INTEGRATION AND POWER-UP

(71) Applicant: GE Precision Healthcare LLC, Waukesha, WI (US)

(72) Inventors: Md Ali Azam, Shorewood, WI (US); Mitchell Smith, Waukesha, WI (US); Nina Balensiefer, Dousman, WI (US); Clayton Franks, Oconomowoc, WI (US)

(73) Assignee: GE Precision Healthcare LLC, Waukesha, WI (US)

(21) Appl. No.: 19/095,321

(22) Filed: Mar. 31, 2025

(51) Int. Cl. A61B 6/58; A61B 6/00; A61B 6/03; A61B 6/46

(52) U.S. Cl. CPC A61B 6/586; A61B 6/032; A61B 6/46; A61B 6/563

(57) ABSTRACT

A computed tomography imaging system includes a gantry and an operator console. The gantry includes first and second circuit boards and a cable electrically connecting the first and second circuit boards. Power is not enabled to the gantry. The operator console is configured to transmit a test request to the first circuit board and receive responses from the first circuit board, detect, based on the response, that there is a fault in at least one of the cable, a seating of the cable in a connector, and the second circuit board, and provide a human perceivable indication of the fault in response to detecting the fault. The test request invokes the first circuit board to transmit a response request to the second circuit board, wait a predetermined time period for a response from the second circuit board, and transmit the response of the second circuit board to the operator console.

### FIELD

The following generally relates to computed tomography (CT), and more particularly to detecting faulty cables, cable connections and/or circuit boards during CT imaging system assembly before full system is integration and powered-up, and is amenable to other imaging systems and/or non-imaging systems.

### BACKGROUND

A computed tomography (CT) imaging system includes a gantry, a rotating frame and a bore. An X-ray tube and one or more rows of detectors are supported on the rotating frame. The X-ray tube is mounted opposite of the one or more rows of detectors, on an opposing side of the bore. The rotating frame, and hence the X-ray tube and one or more rows of detectors, rotate around the bore. The X-ray tube emits X-ray radiation that traverses the bore and is attenuated by subject therein, and the one or more rows of detectors detect X-ray radiation that traverses the subject and generates projection data (line integrals) indicative thereof. The projection data is reconstructed to generate volumetric image data. The voxels of the reconstructed volumetric image data are displayed as two-dimensional (2-D) images and/or three-dimensional (3-D) renderings using gray scale values corresponding to relative radiodensity. The gray scale values reflect the attenuation characteristics of the scanned subject and generally show structure such as anatomical structures within the scanned subject.

The gantry also includes other components such as controllers, a high voltage generator, a power distribution unit, etc., some of which include circuit boards, cables to and/or from the circuit boards, etc. During assembly, such components are assembled in the gantry on the assembly line with no power supplied, e.g., for safety of the assembler and/or the components, system integration incomplete, etc. Once a CT imaging system is assembled, it is moved from the assembly line to a testing bay for staging by a bay technician where power to the gantry is enabled for staging and/or other purposes. Staging process includes further system integration e.g., installing collimator, covers to the gantry, etc. Staging, in general, includes hours of cycle time. Some of the components installed during assembly can pose a significant challenge for the bay technician, requiring the technician to spend hours troubleshooting issues. An example includes issues with cables and/or circuit boards (e.g., a cable not properly seated in a connector, a faulty connector, a faulty cable, a faulty circuit board, a cable seated in the wrong connector, etc.) carried over from the assembly process.

Since the full imaging system is not assembled and there is no way to power the imaging system at the assembly line, detecting certain cable and/or circuit board issues is not readily feasible. After all cables are assembled on the assembly line, a visual inspection can be performed to identify issues such as missing, disconnected, improperly connected, etc. cables. However, visual inspection alone cannot detect all of the aforementioned issues with the cables and/or the circuit boards. Other components e.g., collimator, covers, etc. are installed at the bay by the bay technician, and the imaging system is powered-up for the first time. When a bay technician is confronted with cable and/or the circuit board issue, they have had to de-assemble a part of the CT imaging system, e.g., at least the covers, to access the cables and/or the circuit boards and correct the issue. As such, a very simple issue like reseating a cable on a circuit board has required unnecessary de-assembling part of the imaging system. Unfortunately, this decreases a first pass yield and consumes bay technician time that could be otherwise utilized with staging another CT imaging system, increasing overall cost of the CT imaging system and delaying deployment of the CT imaging system to a customer for use to facilitate diagnosing disease in patients.

In view of at least the foregoing, there is an unresolved need for another approach for detecting certain issues during assembly that mitigate at least the above-noted issues and/or shortcomings.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a computed tomography imaging system includes a gantry. The gantry includes multiple circuit boards, including a first circuit board and a second circuit board. The gantry further includes a cable electrically connecting a first circuit board of the multiple circuit boards and a second circuit board of the multiple circuit boards. Power is not enabled to the gantry. The computed tomography imaging system further includes an operator console. The operator console is configured to transmit a test request to the first circuit board and receive responses from the first circuit board. The test request invokes the first circuit board to transmit a response request to the second circuit board, wait a predetermined time period for a response from the second circuit board, and transmit the response of the second circuit board to the operator console. The operator console is further configured to detect, based on the response, that there is a fault in at least one of the cable, a seating of the cable in a connector, and the second circuit board. The operator console is further configured to provide a human perceivable indication of the fault in response to detecting the fault.

In another aspect, a computer-implemented method includes supplying external power to circuit boards of a gantry of a CT imaging system during assembly of the CT imaging system. Gantry power is not enabled to the gantry during assembly of the CT imaging system. The computer-implemented method further includes transmitting, from an operator console of the CT imaging system, a test request to one of the circuit boards in electrical communication with one or more other circuit boards of the circuit boards via a cable. The test request invokes the one of the circuits boards to transmit a response request to the one of the other circuit boards via the cable. The computer-implemented method further includes receiving, at the operator console, a response of at least the one of the other circuit boards. The computer-implemented method further includes detecting, at the operator console and based on the response, a fault in one of a cable, a seating of a cable and the circuit boards. The computer-implemented method further includes providing an indication indicating the fault is detected.

In another aspect, a system includes a first subsystem with at least multiple circuit boards and a cable electrically connecting a first circuit board of the multiple circuit boards and a second circuit board of the multiple circuit boards, wherein power is not enabled to the first subsystem. The system further includes external power for the at least multiple circuit boards. The system further includes a second subsystem configured to communicate with the multiple circuit boards powered by the external power to detect faulty cables, faulty connections between cables and the multiple circuit boards and faulty circuit boards of the multiple circuit boards.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an imaging system configured for computed tomography (CT) imaging and including a gantry with at least circuit boards, cables, circuit board power and input/output (I/O), and an operator console including at least a cable and/or circuit board test script, a power supply and I/O, in accordance with an embodiment(s) herein.

FIG. 2 schematically illustrates a non-limiting example of the gantry that includes circuit boards, cables connected to the circuit boards, a board power source for the circuit boards and I/O for communication with an external device, in accordance with an embodiment(s) herein.

FIG. 3 schematically illustrates a non-limiting example of the operator console that includes a power supply, I/O for communication with an external device and I/O for a peripheral device, in accordance with an embodiment(s) herein.

FIG. 4 schematically illustrates a non-limiting example in which the gantry of FIG. 2 and the operator console of FIG. 3 are interfaced with each other via corresponding complementary I/O, and the operator console of FIG. 3 is interfaced with an indicator device via corresponding complementary I/O, in accordance with an embodiment(s) herein.

FIG. 5 schematically illustrates a variation of the example described in connection with FIG. 4, in accordance with an embodiment(s) herein.

FIG. 6 schematically illustrates another variation of the example described in connection with FIG. 4, in accordance with an embodiment(s) herein.

FIG. 7 schematically illustrates yet another variation of the example described in connection with FIG. 4, in accordance with an embodiment(s) herein.

FIG. 8 illustrates a non-limiting example of a method, in accordance with an embodiment(s) herein.

FIG. 9 illustrates another non-limiting example of a method, in accordance with an embodiment(s) herein.

FIG. 10 illustrates yet another non-limiting example of a method, in accordance with an embodiment(s) herein.

FIG. 11 illustrates still another non-limiting example of a method, in accordance with an embodiment(s) herein.

### DETAILED DESCRIPTION

Embodiments of the present disclosure will now be described, by way of example, with reference to the figures, in which a system, a method and/or instructions on a computer readable medium for detecting issues with cable and/or circuit boards installed in a gantry of a CT imaging system during assembly of the CT imaging system on the assembly line by an assembly line assembler where power to the gantry is not enabled, i.e., when the imaging system is not yet assembled to the point where power can be supplied thereto, including before the imaging system is moved to a bay and a bay technician installs additional components and powers the imaging system up for the first time. Such issues include faulty cables (e.g., a damaged, severed, pinched, frayed, cut, kinked, etc. wire), faulty cable connections (e.g., a cable connector not seated properly in a circuit board connector, a cable connector seated in an incorrect circuit board connector, a disconnected cable connector, etc.), and/or faulty circuit boards (e.g., a circuit board not functioning according to predetermined operational specifications, etc.). In response to such detection, the assembly line assembler can correct the issue before the CT imaging system is released and moved to a bay for staging by a bay technician.

As discussed above, during assembly of an imaging system such as a CT imaging system on the assembly line by assembly line assembler, gantry power is not enabled, e.g., at least for safety reasons. As such, issues with the cables and/or circuit boards (i.e., faulty cables, faulty cable connections and/or faulty circuit boards) arising during assembly that cannot be visually identified during assembly are not readily detectable during assembly, and are later discovered and/or identified after the CT imaging is moved to a bay for staging by a bay technician. When a bay technician enables power to the gantry, begins staging and detects an issue with the cables and/or circuit boards, the bay technician has had to de-assemble a part of the CT scanner to locate and correct the issue with the cables and/or circuit boards, which lowers the first pass yield and consumes bay technician time, increasing overall cost of the CT imaging system and delaying deployment of a CT imaging system to a customer for use to facilitate diagnosing disease in patient.

The approach described herein detects, during assembly on an assembly line, where power is not enabled to the gantry, by an assembly line assembler cable and/or circuit board issues such as faulty cables, faulty cable connections and/or faulty circuit boards arising during assembly that cannot be visually identified during assembly are not readily detectable during assembly. In one instance, this mitigates passing an assembled CT imaging system and moving the CT imaging system to a bay for a bay technician with faulty cables, faulty cable connections and/or faulty circuit boards. Detecting such assembly issues during assembly allows for correcting the issue during assembly, allowing the bay technician to focus their time staging CT imaging systems, and not adding time for de-assembling, troubleshooting and correcting assembly-time issues, nor adding cost, nor delaying deployment of a CT imaging system to a customer.

As described in greater detail below, in one instance power external to the gantry is provided to the circuit boards in the gantry and communication is established between a computing system (e.g., the operator console, etc.) and the circuit boards. A cable and/or circuit board test script executing at the computing system communicates with at least one circuit board of the gantry, transmitting a test request to the at least one circuit board of the gantry. The at least one circuit board of the gantry, in response thereto, transmits a response request to other circuit boards in the gantry, waits for a predetermined time period during which it receives responses from the circuit boards, and provides the responses to the cable and/or circuit board test script. The cable and/or circuit board test script evaluates the responses to detect possible cable and/or circuit board issues such as faulty cables, faulty cable connections and/or faulty circuit boards. This allows the assembly line assembler to correct such issues before the CT imaging system is moved to a bay for staging by a bay technician.

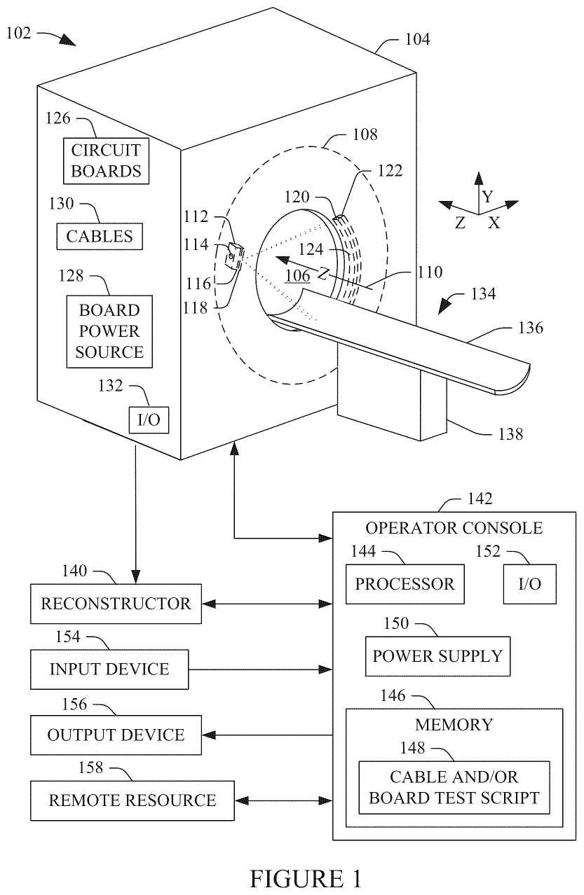

Initially referring to FIG. 1, a non-limiting example of an imaging system 102 configured for computed tomography (CT) imaging is schematically illustrated. The imaging system 102 includes a gantry 104 with a bore 106 and a rotating frame 108. The rotating frame 108 is rotatably supported by the gantry 104, e.g., via a bearing (e.g., a slip ring, etc.) or the like, and is configured to rotate around the bore 106 about a rotational or z-axis 110, which extends through a center of rotation (e.g., a center of the bore 106, i.e., an isocenter). In some instances, the gantry 104 can be configured to tilt through the z-axis 110. A rotating frame controller (not visible) is configured to control rotation (and tilt, if available) of the rotating frame 108, including no rotation.

The rotating frame 108 carries components utilized in the generation, emission and detection of X-rays. For instance, an X-ray source assembly 112 is supported by the rotating frame 108 and rotates in coordination with the rotating frame 108. The X-ray source assembly 112 includes an X-ray source 114 such as an X-ray tube and/or other source that generates and/or emits X-ray radiation. The X-ray assembly 112 further includes or is coupled to a filter 116, such as a filter that characterizes a radiation dose profile. The X-ray assembly 112 further includes or is coupled to a collimator 118 that shapes the X-ray radiation to form a generally fan, wedge, cone, etc. shaped beam that traverses the bore 106. An X-ray controller is configured to control components of the X-ray assembly 112, including the X-ray source 114 (e.g., such as kVp, mAs, etc.), the collimator 118 (e.g., a width of the X-ray beam), etc.

Additionally, a detector array 120 and a Data Acquisition System (DAS) 122 are supported by the rotating frame 108. The detector array 120 is supported by the rotating frame 108 along an arc, opposite the X-ray source assembly 112, across the bore 106, and rotate in coordination with the rotating frame 108, including the X-ray source 114. The detector array 120 includes a one-dimensional (1-D) and/or a two-dimensional (2-D) array of rows of X-ray radiation sensitive detector elements 124. The detector array 120 is in electrical communication with the DAS 122. A DAS controller (not visible) controls the detector array 120, the X-ray radiation sensitive detector elements 124, the DAS 122, etc. Other components are also supported by the rotating frame 108. For example, one side of the slip ring, a cooling system, a high voltage generator, etc. are supported by the rotating frame 108.

The gantry 104 further includes other components, some on the rotating and others not on the rotating frame 108. Examples of such components include a high voltage generator, controls integrated in the cover, patient positioning lights, fans/blowers, interfaces to devices outside of the gantry 104, etc. Some of these components include circuit boards 126. A board power source 128 is configured to receive power and supply power to the circuit boards 126. Cables 130 provide electrical pathways with the circuit boards 126. In one instance, the circuit boards 126 and the cables 130 include complementary sides of electro-mechanical connectors, which detachably engage and/or are affixed. In another instances, some of the cables 130 are directly connected (i.e., no electromechanical connectors) to some of the circuit boards 126, e.g., via solder, etc. Input/Output (I/O) 132 provides a communication path with one or more systems and/or devices outside of the gantry 104.

During assembly of the imaging system 102 on the assembly line by an assembly line assembler, electrical power is not enabled to the gantry 104, e.g., at least for safety purposes. Without power, issues with the cables 130 and/or circuit boards 126, such as faulty cables, faulty cable connections and/or faulty circuit boards, during assembly are not readily detectable during assembly, and are later discovered and/or identified after the CT imaging system 102 is moved to a bay for staging by a bay technician. When a bay technician detects such an issue with the cables 130 and/or circuit boards 126, they may have to de-assemble a part of the CT imaging system to identify and address the issue, which can be time intensive, increasing overall cost and delaying deployment of a CT imaging system to a customer for use to facilitate diagnosing disease in patient. As described in greater detail below, in one instance the approach described herein detects such issues during assembly when power is not enabled to the gantry 104, allowing detected issues to be addressed prior to moving the imaging system 102 for staging.

The imaging system 102 further includes a table 134. The table 134 includes a cradle 136 moveably coupled to a frame/base 138. In one instance, the cradle 136 is slidably coupled to the frame/base 138 via a bearing or the like, and a drive system (not visible) including a motor, a lead screw, and a nut (or other drive system) translates the cradle 136 along the frame/base 138 into and out of the bore 106 for horizontal motion, and the frame/base 138 includes a drive system (not visible) including a mechanism for vertical or diagonal motion. The cradle 136 is configured to support a subject in the bore 106 for loading, scanning, and/or unloading. A table controller (not visible) controls the drive system of the table 134.

For an axial (step and shoot) scan, the cradle 136 is positioned at a static position for each integration period and moves between integration periods. For a helical scan, the rotating frame 108 rotates in coordination with the cradle 136 moving along the Z-axis 110, and active X-ray radiation sensitive detector elements 124 of the detector array 120 detect X-ray radiation over consecutive arc segments (integration periods) with each revolution and generate respective signals. For each arc segment, the DAS 122 processes each signal and generates projection data.

A reconstructor 140 reconstructs the projection data and generates 3-D volumetric image data for a helical scan and/or an individual axial (2-D) slice for an axial step and shoot scan (which can be used in combination to generate volumetric image data). The 3-D volumetric image data and/or 2-D slices thereof, and/or the individual axial images can be visually presented, filmed, etc. Examples of suitable reconstruction algorithms include filtered back projection (FBP), advanced statistical iterative reconstruction (ASIR), conjugate gradient (CG), maximum likelihood expectation maximization (MLEM), model-based iterative reconstruction (MBIR), and/or other reconstruction algorithm.

A computing system 142 serves as an operator console of the imaging system 102. The computing system 142 includes a computer, a workstation, server, etc. The computing system 142 includes a processor 144 such as a microprocessor (mP), a central processing unit (CPU), graphics processing unit (GPU), etc., and a computer readable medium (memory) 146, which includes non-transitory medium and excludes transitory medium (signals, carrier waves, and the like). The computer readable medium 146 at least includes a cable and/or board test script 148. As described in greater detail below, in one instance the cable and/or board test script 148 is configured to detect issues with the cables 130 and/or the circuit boards 126 during assembly on the assembly line by an assembly line assembler where power is not enabled to the gantry 104.

The computing system 142 further includes a power supply 150. The power supply 150, in one instance, is configured to supply power to electrical components external to the computing system 142 such as one or more components of the gantry 104, e.g., the board power source 128, etc. The computing system 142 further includes input/output (I/O) 152. An input device 154 includes a keyboard, mouse, touchscreen, microphone, etc. The computing system 142 and the input device 154 are in electrical communication through the I/O 152 and/or otherwise. An output device 156 includes a human readable device such as a display monitor or the like. The computing system 142 and the output device 156 are in electrical communication through the I/O 152 and/or otherwise. The I/O 152 further includes I/O for interfacing with the I/O 132 of the gantry 104 (e.g., complementary network infaces, etc.), external devices (e.g., complementary Universal Serial Bus (USB), etc. interfaces).

A remote resource 158 includes one or more of a server, a workstation, a Radiology Information System (RIS), a Hospital Information System (HIS), an Electronic Medical Record (EMR), a Picture Archiving and Communication System (PACS) for storing information, a PACS further configured with image viewing and/or manipulating software, cloud resources with shared remote data storage and/or computing power including resources distributed over data centers, etc. The computing system 142 and the remote resource 158 are in communication via wired and/or wireless technologies. Such communication can be through Digital Imaging and Communications in Medicine (DICOM), Health Level Seven (HL7), etc. formats and protocols.

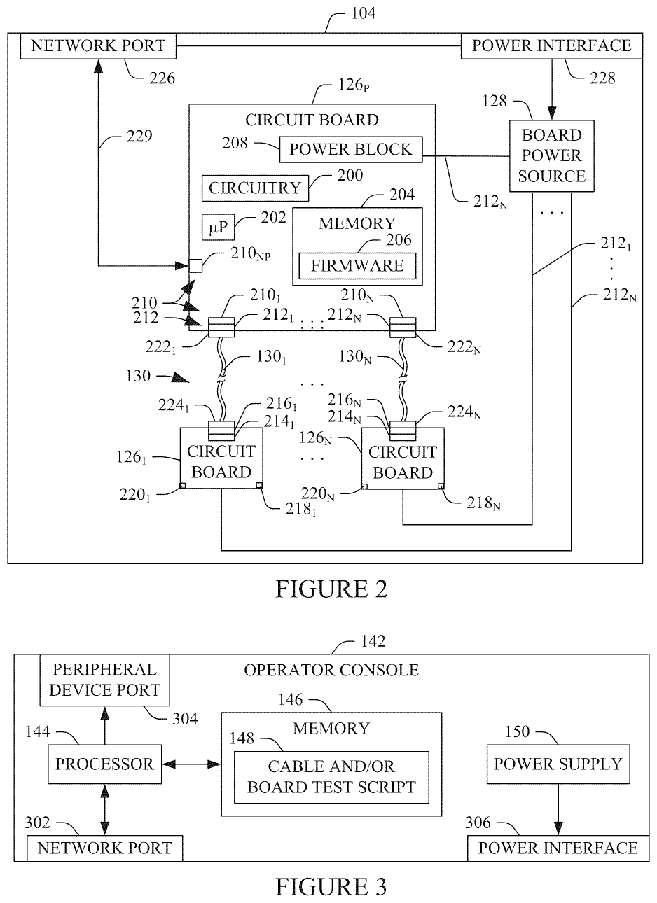

Turning to FIG. 2, an example configuration of the circuit boards 126, the board power source 128, the cables 130 and the I/O 132 of the gantry 104 is schematically illustrated.

In this example, the circuit boards 126 includes a plurality of circuit boards, including a circuit board 126_{P}, a circuit board 126₁, . . . , and a circuit board 126_{N}, where N is a positive integer. In this example, the circuit board 126_{P} is considered a primary, master, etc. circuit board in that it is configured to communicate with the circuit board 126₁, . . . , the circuit board 126_{N}, and/or other components internal to and/or outside of the gantry 104. Examples of such communication with the circuit boards 126 include a transmit signal (e.g., a command, an instruction, etc.) to one or more of the circuit boards 126₁, . . . , the circuit board 126_{N}, receive a signal from one or more of the circuit boards 126₁, . . . , the circuit board 126_{N} (e.g., a response, a result, etc.), etc. In another instance, more than one of the circuit boards 126 is configured to communicate as such, alternatively and/or concurrently.

The circuit board 126_{P} includes electrical circuitry 200, a microprocessor (mP) 202 and a memory 204. The memory 204 stores firmware 206. In this example, the memory 204 of the circuit board 126_{P} at the time of installation during assembly does not include the firmware 206. The firmware 206 is encoded in the memory 204, e.g., via flashing the memory 204, i.e., programming the memory 204 with the firmware 206. The mP 202 is configured to execute the firmware 206 in the memory 204. In general, the firmware 206 includes the instructions and/or data to utilize the circuitry 200 to provide the functionality of the circuit board 126_{P}. The circuit board 126_{P} further includes a power block 208. The power block 208 receives power from the board power source 128 and supplies operational power to components of the circuit board 126_{P}.

The circuit board 126_{P} further includes I/O 210, including an I/O 210_{NP}, an I/O 210₁, . . . , and an I/O 210_{N}. In this example, the I/O 210_{NP} is a network port. For example, in one instance the I/O 210_{NP} is at least configured for Ethernet. In this instance, the I/O 210_{NP} may include a wired and/or wireless interface. For example, in one instance the I/O 210_{NP} includes an RJ45 receptacle for receiving an RJ45 plug of an Ethernet cable and/or a Wi-Fi interface. Additionally, or alternatively, the I/O 210_{NP} includes another wireless network interface, e.g., Bluetooth, Zigbee, etc. Additionally, or alternatively, the I/O 210_{NP} includes a fiber optic network interface. Other network technologies are also contemplated herein. Each of the I/O 210₁, . . . , and the I/O 210_{N} includes a corresponding electro-mechanical connector 212, including an electro-mechanical connector 212₁, . . . , and an electro-mechanical connector I/O 212_{N}.

The circuit board 126₁, . . . , and the circuit board 126_{N} respectively include an I/O 214₁, . . . , and an I/O 214_{N}. Similar to the circuit board 126_{P}, the I/O 214₁, . . . , and the I/O 214_{N} respectively include an electro-mechanical connector 216₁, . . . , and an electro-mechanical connector I/O 216_{N}. Also similar to the circuit board 126_{P}, the circuit board 126₁, . . . , and the circuit board 126_{N} include power block 218₁, . . . , and a power block 218_{N}, circuitry 222₁, . . . , and circuitry 222_{N}, etc. One or more of the circuit board 126₁, . . . , and the circuit board 126_{N} may include the same, additional, more, less, etc. components relative to the circuit board 126_{P} and/or other circuit boards 126. For sake of brevity and/or clarity, all of the possible components of the circuit boards 126 are not schematically illustrated. However, the skilled artisan would readily understand the types of elements of such components and/or their functionality.

The cables 130 include a cable 130₁, . . . , and a cable 130_{N}. Each of the cables include a set of connectors, one at each end. For example, the cable 130₁ includes a electro-mechanical connector 222₁ at one end of the cable 130₁ and a electro-mechanical connector 224₁ at an opposing end of the cable 130₁, . . . , and the cable 130_{N} includes a electro-mechanical connector 222_{N} at one end of the cable 130_{N} and a electro-mechanical connector 224_{N} at an opposing end of the cable 130_{N}.

The cable 130₁ is configured to provide an electrical communications path between the electro-mechanical connector 222₁ and the electro-mechanical connector 212₁. That is, the electro-mechanical connector 222₁ and the electro-mechanical connector 212₁. are complementary electro-mechanical connector, e.g., male type (i.e., pins, prongs, etc.) and female type (i.e., sockets, receptacle, etc.) connectors. During assembly, if the electro-mechanical connector 222₁ is not properly seated (e.g., inserted and secured) in the electro-mechanical connector 212₁, the electrical communications path established thereby may be compromised during bay staging.

The cable 130₁ is further configured to provide an electrical communications path between the electro-mechanical connector 224₁ and the electro-mechanical connector 216₁. Likewise, that is, the electro-mechanical connector 224₁ and the electro-mechanical connector 216₁. are complementary electro-mechanical connectors. Likewise, if the electro-mechanical connector 224₁ is not properly seated in the electro-mechanical connector 216₁ during assembly, the electrical communications path established thereby may be compromised during bay staging.

The cable 130_{N} is further configured to provide an electrical communications path between the electro-mechanical connector 222_{N} and the electro-mechanical connector 212_{N}. Likewise, that is, the electro-mechanical connector 222_{N} and the electro-mechanical connector 212_{N}. are complementary electro-mechanical connectors. Likewise, if the electro-mechanical connector 222_{N} is not properly seated in the electro-mechanical connector 212_{N} during assembly, the electrical communications path established thereby may be compromised during bay staging.

The cable 130_{N} is further configured to provide an electrical communications path between the electro-mechanical connector 224_{N} and the electro-mechanical connector 216_{N}. Likewise, that is, the electro-mechanical connector 224_{N} and the electro-mechanical connector 216_{N} are complementary electro-mechanical connectors. Likewise, if the electro-mechanical connector 224_{N} is not properly seated in the electro-mechanical connector 216_{N} during assembly, the electrical communications path established thereby may be compromised during bay staging.

The I/0 132 (FIG. 1) of the gantry 104 includes a network port 226. The network port 226 is in electrical communication with the network port (I/O) 210_{NP} via a network communications path 229. The network port 226 further includes a network interface for a device external to the gantry104, including the types of networks discussed in connection with the network port 210_{NP} above. For example, in one instance the network port 226 is at least configured for Ethernet and includes an RJ45 receptacle for receiving an RJ45 plug of an Ethernet cable.

The gantry 104 further includes a power interface 228. The power interface is configured to receive power external to the gantry 104 for the board power source 128 (and supporting componentry like the network port 226 in this example) and supply operational power to the circuit boards 126 (and the supporting componentry). It is to be understood that in this example the board power source 128 does not provide general power to the gantry 104 for the other electrical-based components of the gantry 104. Rather, the board power source 128 is configured to supply power to the circuit boards 126ₚ (and supporting componentry), e.g., during assembly where gantry power is not enabled at least for cable and/or board testing purposes.

In one instance, the power interface 228 includes a recessed plug and/or a socket configured for engagement with a complementary socket and/or plug of the external power source through a power cable or the like. The interface type and electrical rating depend on the country of deployment. For example, in the United States (US), a National Electrical Manufacturers Association (NEMA) based interface with an electrical rating of 110-120 volts alternating current (110-120 VAC) and 60 Hertz (60 Hz) is suitable. In another example, in the Europe, an International Commission on Rules for the Approval of Electrical Equipment (CEE) based interface with an electrical rating of 220-240 VAC and 50 Hz is suitable.

Next at FIG. 3, an example configuration of the I/O 152 (FIG. 1) of the operator console 142 is schematically illustrated.

With regard to the operator console 142, the I/O 152 at least includes a network port 302 and a peripheral device port 304. The network port 302 includes a network interface such as the types of networks discussed in connection with the network port 210_{NP} and the network port 302 above, e.g., an RJ45 receptacle for receiving an RJ45 plug of an Ethernet cable. The peripheral device port 304 includes an interface for a peripheral device. An example of such a port is a USB based port such as USB-A, USB-C, etc. and/or other port. As described in greater detail below, an example peripheral device includes an indicator device, which is utilized by the cable and/or board test script to indicate results of cable and/or circuit board tests such as pass or fail.

A power interface 306 is configured to receive electrical power from the power supply 150 and supply power for an external device, such as the circuit boards 126 of the gantry 104 (FIGS. 1 and 2) and/or other external device, via the power interface 228 (FIG. 2) and the board power source 128 (FIGS. 1 and 2). Similar to the power interface 306 of the gantry104, in one instance the power interface 306 includes a recessed plug and/or a socket configured for engagement with a complementary socket and/or plug, where the interface type and electrical rating depend on the country of deployment, e.g., a NEMA type interface with 110-120 VAC and 60 Hz in the US, a CEE based interface with 220-240 VAC and 50 Hz in Europe, etc.

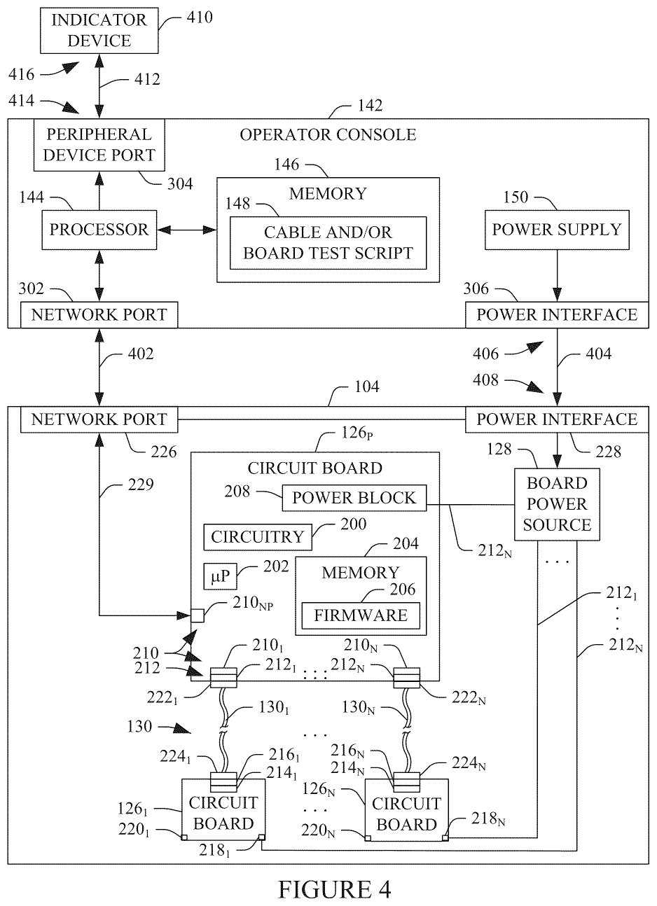

FIG. 4 schematically illustrates an example employing the configuration of the gantry 104 described in connection with FIG. 2 and the configuration of operator console 142 (along with a peripheral device in electrical communication therewith) described in connection with FIG. 3. For this example, the imaging system 102 is being assembled on the assembly line by an assembly line assembler, and power is not enabled to the gantry 104. Furthermore, the cable and/or board test script 148 is configured to detect instances in which a cable of the cables 130 is faulty, a cable of the cables 130 is not seated properly and/or is seated in an incorrect connector, circuit board of the circuit boards 126 is faulty, etc.

A network cable 402 provides a communications path between the operator console 142 and the gantry 104 via the network port 302 of the operator console 142 and the network port 226 of the gantry 104. Where the network port 302 of the operator console 142 and the network port 226 of the gantry 104 include Ethernet ports, the cable 402 includes a category X (CAT X) based cable such as a CAT 5, a CAT 6, etc. based cable with corresponding plugs on both ends, which interface with RJ45 ports of the network port 302 of the operator console 142 and the network port 226 of the gantry 104.

A power cable 404 provides a path for electrical power between the operator console 142 and the gantry 104 via the power interface 306 of the operator console 142 and the power interface 228 of the gantry 104. Where the imaging system 102 is being deployed in the US, an end 406 of the power cable 404 includes a socket type connector and an opposing end 408 of the power cable 404 includes a prong type connector. An example of the power cable is a 120 VAC, 60 Hz, NEMA 5 -15 three (3) prong power cable.

A peripheral indicator device 410 includes a feedback device such as visual (e.g., a light, etc.) feedback device, an audible (e.g., a sound, etc.) feedback device, a haptic (e.g., a vibration, etc.) feedback device, and/or other feedback device. In one instance, the indicator device 410 is a USB based (e.g., USB-A, USB-C, etc.) device configured to receive electrical power from and communicate with a host device, such as the imaging system 102 and/or other device.

A peripheral device cable 412 provides a communications path between the indicator device 410 and the peripheral device port 304 of the operator console 142. Where the peripheral device port 304 includes a USB port, an end 414 of the peripheral device cable 412 includes a complementary USB connector configured to connect to the peripheral device port 304 of the operator console 142. An opposing end 416 of the peripheral device cable 412 either includes a USB connector or is hardwired with the indicator device 410.

Again, during assembly of the imaging system 102 on an assembly line by an assembly line assembler, electrical power is not enabled to the gantry 104, and the assembly line assembler installs at least the circuit boards 126 and the cables 130. Without such power, issues with the cables 130 and/or circuit boards 126, such as faulty cables, faulty cable connections and/or faulty circuit boards, during assembly are not readily detectable. As described next, in one instance the approach described herein detects such issues with the cables 130 and/or circuit boards 126 during assembly on the assembly line when power is not enabled to the gantry 104, allowing detected issues with the cables 130 and/or circuit boards 126 to be addressed prior to moving the imaging system 102 for staging.

With the approach disclosed herein, the assembly line assembler provides power to the operator console 142, e.g., plugs the operators console 142 into a main power line such as a wall or other live receptacle. The assembly line assembler further connects the network cable 402 to the network port 302 of the operator console 142 and the network port 226 of the gantry104. The assembly line assembler further connects the power cable 404 to the power interface 306 of the operator console 142 and the power interface 228 of the gantry104. If not already connected, the assembly line assembler further connects the peripheral device cable 412 to at least the peripheral device port 304 of the operator console 142, and further to the indicator device 410 where the peripheral device cable 412 is not hardwired to the indicator device 410.

Electrical power from power interface 228 supplies operational power to the network port 226 of the gantry104. Electrical power from power interface 228 further supplies operational power to the board power source 128 of the gantry104. The board power source 128 of the gantry 104 supplies power to the circuit boards 126, including the circuit board 126_{P}, the circuit board 126₁, . . . , and the circuit board 126_{N}. Powering the circuit board 126_{P}, powers the network interface 210_{NP} of the circuit board 126_{P}, establishing a network connection between the network port 302 of the operator console and the network interface 210_{NP} of the circuit board 126_{P} via the network port 226 and the network cable402.

The assembly line assembler executes the cable and/or board script 148 via a keyboard of the input device 154 (FIG. 1), e.g., via a common prompt and/or other user interface. In one instance, the executing cable and/or board script 148 flashes the memory 204 of the circuit board 126_{P} to program the memory 204 with the firmware 206. In another instance, another program and/or device is employed to flash the memory 204 with the firmware 206. In yet another instance, the memory 204 is pre-programmed with the firmware 206.

The executing cable and/or board script 148 transmits a test request over the network connection (i.e., the network port 302, the network cable 402, the network port 226, the network path 229, and the network interface 210_{NP}) to the circuit board 126_{P}. The circuit board 126_{P}, in response to receiving the test request, transmits a response request to each of the circuit boards of the circuit boards 126 that the circuit board 126_{P} is configured to communicate with and test. In general, the response request invokes a response from each circuit board of the circuit boards 126 that receives the response request.

In this example, the cables 130 between the circuit board 126_{P} and the other circuit board of the circuit boards 126 includes a network cable (e.g., Ethernet, optical, etc.), and the response request includes a ping or the like. With a ping, an Internet Control Message Protocol (ICMP) echo request is transmitted to each of the circuit boards 126 (e.g., via an Internet Protocol (IP) address). Where a circuit board of the of the circuit boards 126 receives the ping and is properly functioning, the circuit board responds with an ICMP echo reply. Other signals are transmitted for instances where the connection is not an Ethernet based connection. For example, where an electro-mechanical connector includes a serial port (e.g., a communication (COM) port, etc.), a loopback test, serial port monitoring software, and/or other approach can be utilized.

The circuit board 126_{P} forwards responses of the circuit boards 126 to the executing cable and/or circuit board test script 148 at the operator console 142 via the network connection (i.e., the interface 210_{NP}, the network path 229, the network port 226, the network cable 402, and the network port 302). In general, a response as utilized herein includes an actual response (i.e., an echo reply) as well as no response (e.g., from a circuit board of the circuit boards 126 not receiving the ping and/or not able to respond to the ping). In one instance, the circuit board 126_{P} provides the executing cable and/or circuit board test script 148 with circuit board identification information (e.g., IP address, etc.) along with the responses (including no response) of the circuit boards 126.

The executing cable and/or board script 148 evaluates the response information and detects instances in which there is an issue with the cables 130 and/or the circuit boards 126. For example, the executing cable and/or board script 148 detects there is a potential issue where a circuit board did not respond with an echo reply. In another example, for instance, where the information further includes timing information of a response, the executing cable and/or board script 148 may determine that there is a potential issue where the timing of an echo reply is outside of a predetermined time period for the echo reply.

In the illustrated example, the executing cable and/or board script 148 employs the indicator device410 to notify the assembly line assembler of results or a status of the test. In one instance, the indicator device 410 is activated (e.g., where the indicator device 410 is a light, the light turns on, flashes on and off, etc.) when the testing passes, and not activated (e.g., where the indicator device 410 is a light, the light remains off) when the testing fails. In another instance, the opposite approach is employed. That is, the indicator device 410 is not activated when the testing passes, and is activated when the testing fails.

Where the indicator device 410 indicates there is an issue, the assembly line assembler can view the information via display monitor of the output device 156 (FIG. 1) to identify which of the circuit boards 126 did not respond. In one instance, such information is stored in a log or other file. In one instance, this includes storing the information as a list, a flat file, and/or otherwise and includes at last the circuit board identification and an indication of whether a response was received. In another instance, additional information such as a circuit board name, serial number, etc. is further included and used for locating cables and/or circuit boards to check.

In any instance, the assembly line assembler can address the issue with the cables 130 and/or circuit boards 126 prior to passing the imaging system 102 to be moved to a bay for staging by a bay technician. As discussed herein, during assembly of a CT imaging system (such as the imaging system 102) on the assembly line, electrical power is not enabled to the gantry 104, and by detecting and correcting such issues during assembly, the bay technician can focus their time on staging instead of troubleshooting and correcting issues arising during assembly, improving first pass yields and/or mitigating additional cost and/or additional time to field deployment.

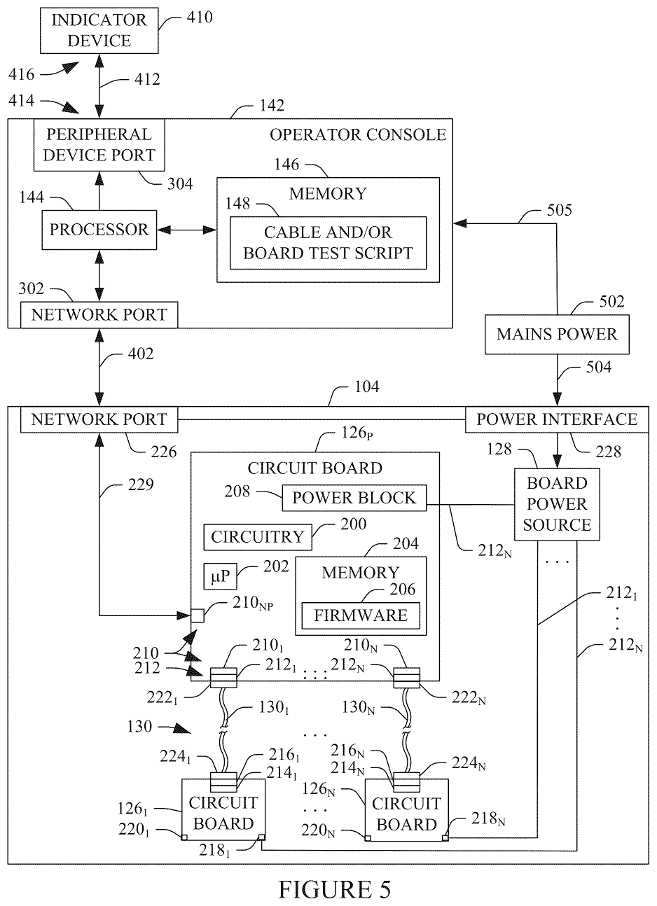

FIG. 5 schematically illustrates a variation of the example described in connection with FIG. 4. In this variation, the electrical power is provided to the power interface 228 of the gantry 104 and the operator console 142 via a mains power source 502 (e.g., a wall receptacle, etc.). A power cable 504 provides a path for electrical power between the mains power source 502 and the gantry 104 via the power interface of the gantry 104. A power cable 505 provides a path for electrical power between the mains power source 502 and the operator console 142. Where the imaging system 102 is being deployed in the US, the power cables 504 and 505 include a plug and socket NEMA-type connector, with a rating for 120 VAC, 60 Hz.

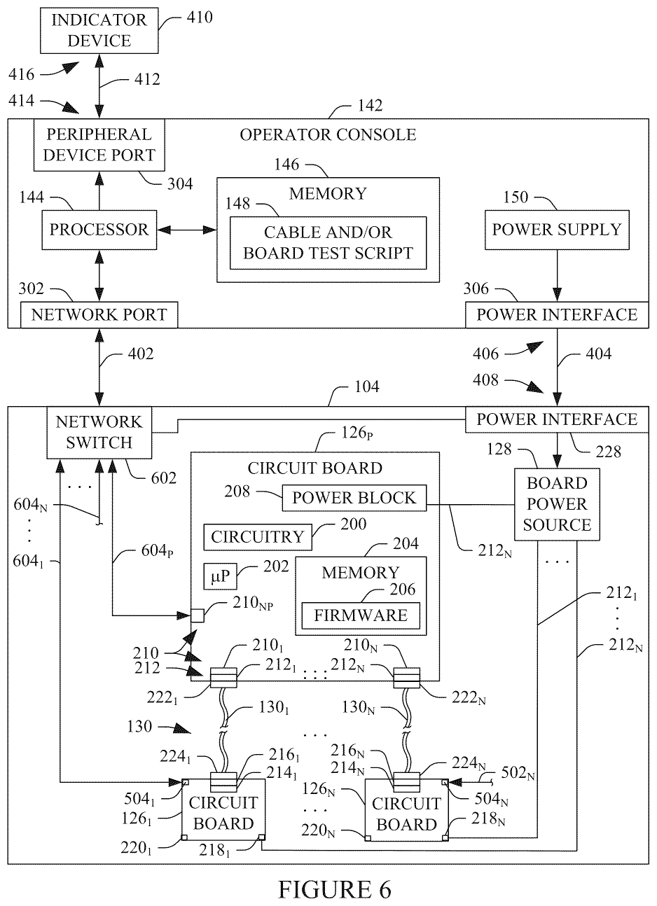

FIG. 6 schematically illustrates another variation of the example described in connection with FIG. 4. In this variation, the gantry 104 includes a network switch 602 that is in electrical communication with more than one of the circuit boards 126. In this example, the network switch 602 is in electrical communication with the circuit board 126_{P} via a network connection 604_{P}, the circuit board 126₁ via a network connection 604₁, . . . , and the circuit board 126_{N} via a network connection 504_{N}, In this instance, the cable and/or circuit board test script 148 can communicate individually with each of the circuit boards 126 interfaced with the network switch 602 and transmit a response request signal directly to a circuit board of the circuit boards 126 and receive a response signal directly from the circuit board of the circuit boards 126.

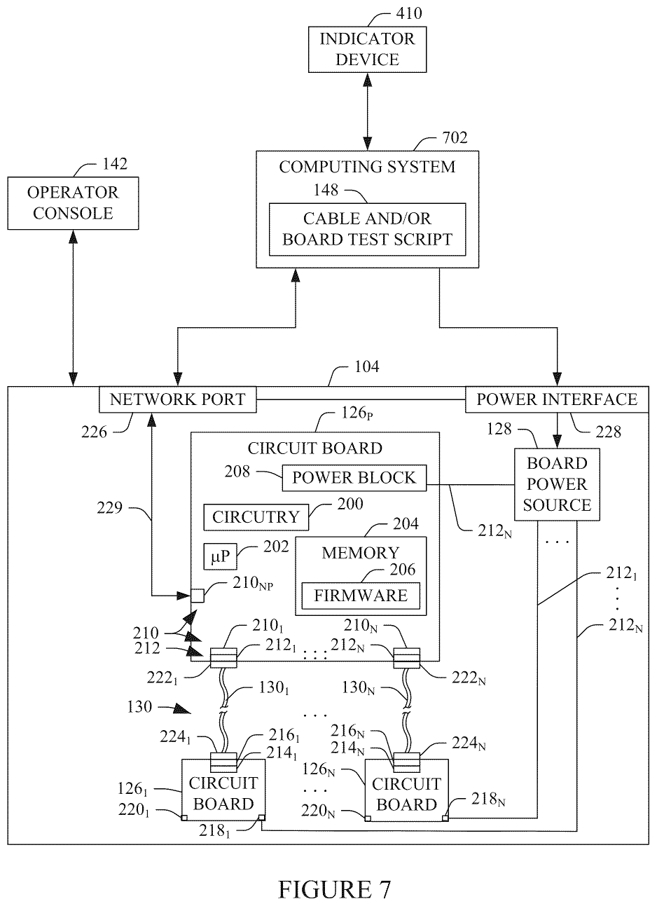

FIG. 7 schematically illustrates yet another variation of the example described in connection with FIG. 4. In this variation, the cable and/or circuit board test script 148 (and the power supply 150, the network interface 226 and the peripheral device port 304, which are not repeated in FIG. 7 for sake of brevity and clarity) are part of a computing system 702, and the indicator device 410 is interfaced with the computing system 702. With this example, the assembly line assembler uses the computing system 702 to detect faulty cables, faulty cable connections and/or faulty circuit boards 126, as discussed herein and/or otherwise.

In the above examples, the approach is described in connection with an imaging system during assembly of the imaging system where power is not enabled to the gantry of the imaging system. In another instance, the approach described herein is employed with an already assembled imaging system that has passed assembly and is not on the assembly line, but power is not supplied to the gantry, either intentionally (e.g., the power is turned off, etc.) and/or other reasons such as a power outage, is power being installed, etc.

In the above examples, the approach is described in connection with a medical imaging system. More generally, the approach described herein is amenable to non-imaging systems. In one instance, in this context, a first subsystem includes at least multiple circuit boards and a cable electrically connecting a first circuit board of the multiple circuit boards and a second circuit board of the multiple circuit boards. Power is not enabled to the first subsystem, and external power is provided for the least multiple circuit boards of the first subsystem. A second subsystem is configured to communicate with the multiple circuit boards powered by the external power to detect faulty cables, faulty connections between cables and the multiple circuit boards and faulty circuit boards of the multiple circuit boards.

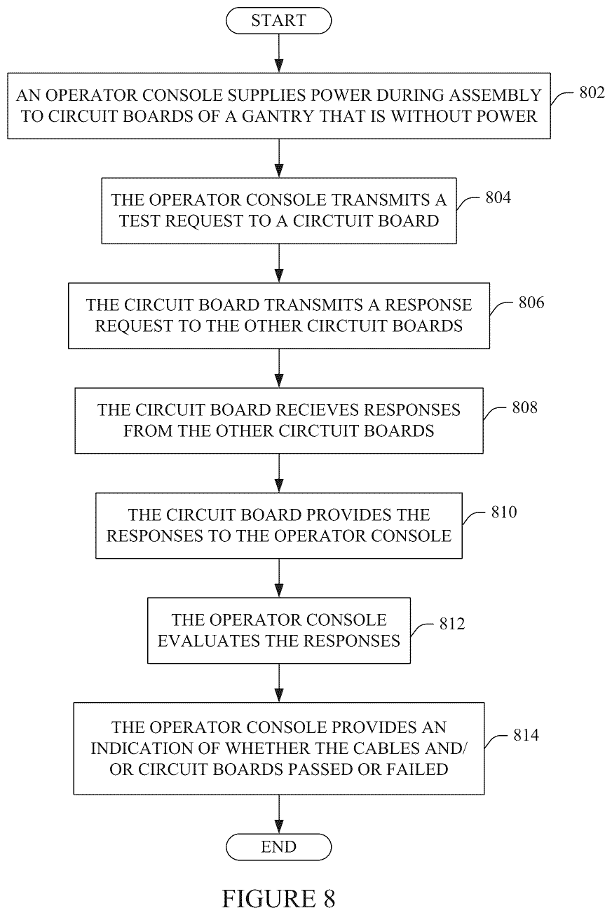

Next at FIG. 8, a non-limiting example of a method is illustrated. It is to be appreciated that the ordering of the acts in the method is not limiting. As such, other orderings are contemplated herein. In addition, one or more acts may be omitted, and/or one or more additional acts may be included.

At 802, circuit board power from an operator console of an imaging system is supplied to one or more circuit boards of a gantry of the imaging system during assembly of the imaging system where gantry power is not enabled, as described herein and/or otherwise. For example, in one instance the power supply 150 (FIGS. 1, 3, 4, and 6) of the operator console 142 supplies power for powering the circuit boards 126 of the gantry 104.

At 804, the operator console transmits a test request to at least one of the circuit boards of the gantry, as described herein and/or otherwise. For example, in one instance the cable and/or board test script 148 executing in the operator console 142 transmits a test request to at least the circuit board 126_{P} in the gantry 104 of the imaging system 102.

At 806, the at least one circuit board of the gantry of the CT imaging system transmits a response request to one or more of the other circuit boards of the gantry, as described herein and/or otherwise. For example, in one instance at least the circuit board 126_{P} in the gantry 104 of the imaging system 102 transmits a response request to one or more of the circuit board 126₁, . . . , and the circuit board 126_{N}.

At 808, the at least one circuit board of the gantry of the CT imaging system receives responses from the one or more circuit boards of the gantry, as described herein and/or otherwise. For example, in one instance at least the circuit board 126_{P} in the gantry 104 of the imaging system 102 waits a predetermined time period for responses from the one or more of the circuit board 126₁, . . . , and the circuit board 126_{N}. This includes an actual response signal as well as no response.

At 810, the at least one circuit board of the gantry transmits responses from the one or more circuit boards of the gantry to the operator console, as described herein and/or otherwise. For example, in one instance the at least the circuit board 126_{P} in the gantry 104 of the imaging system 102 transmits responses from the one or more of the circuit boards 126₁, . . . , and the circuit board 126_{N} along with circuit board identification information to the operator console 142.

At 812, the operator console evaluates the responses to detect issues with the cables and/or circuit boards, as described herein and/or otherwise. For example, where a particular circuit board (identified via identification information) of the circuit boards 126 did not transmit a response, the operator console 142 determines the circuit board is associated with a faulty cable, a faulty cable connection and/or is a faulty circuit board. In other instances, other information may also be utilized to make this determination.

At 814, the operator console notifies the assembly line assembler that there is likely a faulty cable, a faulty cable connection and/or a faulty circuit board, as described herein and/or otherwise. For example, in one instance the cable and/or board test script 148 employs the indicator device 410 to visually, audibly and/or haptically indicate that a cable and/or circuit board test failed and that at least one of the cables 130, the circuit boards 126 and/or a connection therebetween is likely faulty.

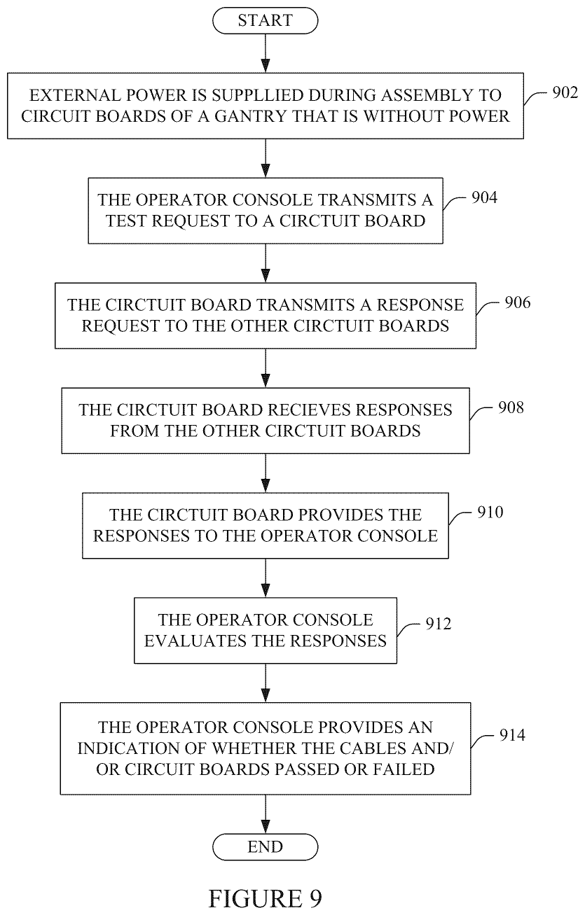

Next at FIG. 9, a non-limiting example of another method is illustrated. It is to be appreciated that the ordering of the acts in the method is not limiting. As such, other orderings are contemplated herein. In addition, one or more acts may be omitted, and/or one or more additional acts may be included.

At 902, power external to the imaging system is supplied to one or more circuit boards of a gantry of the imaging system during assembly of the imaging system where gantry power is not enabled, as described herein and/or otherwise. For example, in one instance the mains power 502 (FIG. 5) supplies power for powering the circuit boards 126 of the gantry 104. At 904, the operator console transmits a test request to at least one of the circuit boards of the gantry, as described herein and/or otherwise. At 906, the at least one circuit board of the gantry of the CT imaging system transmits a response request to one or more of the other circuit boards of the gantry, as described herein and/or otherwise.

At 908, the at least one circuit board of the gantry of the CT imaging system receives responses from the one or more circuit boards of the gantry, as described herein and/or otherwise. At 910, the at least one circuit board of the gantry transmits responses from the one or more circuit boards of the gantry to the operator console, as described herein and/or otherwise. At 912, the operator console evaluates the responses to detect issues with the cables and/or circuit boards, as described herein and/or otherwise. At 914, the operator console notifies the assembly line assembler that there is likely a faulty cable, a faulty cable connection and/or a faulty circuit board, as described herein and/or otherwise.

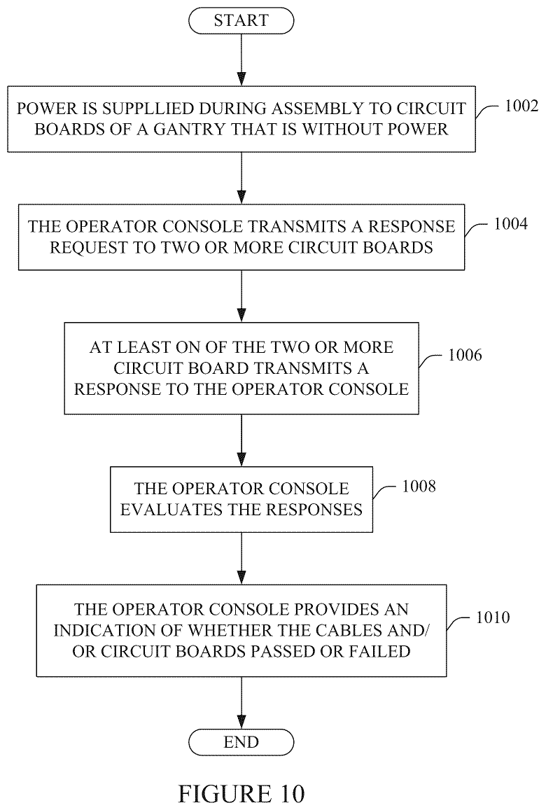

Next at FIG. 10, a non-limiting example of yet another method is illustrated. It is to be appreciated that the ordering of the acts in the method is not limiting. As such, other orderings are contemplated herein. In addition, one or more acts may be omitted, and/or one or more additional acts may be included.

At 1002, power is supplied to one or more circuit boards of a gantry of the imaging system during assembly of the imaging system where gantry power is not enabled, as described herein and/or otherwise. For example, in one instance the power supply 150 (FIGS. 1, 3, 4, and 6) of the operator console 142 supplies power for powering the circuit boards 126 of the gantry 104. In one instance, the mains power 502 (FIG. 5) supplies power for powering the circuit boards 126 of the gantry 104.

At 1004, the operator console transmits a test request to two or more of the circuit boards of the gantry, as described herein and/or otherwise. For example, in one instance the cable and/or board test script 148 executing in the operator console 142 transmits a response request to two or more the circuit boards 126_{P}, the circuit board 126₁, . . . , and the circuit board 126_{N}. in the gantry 104 of the imaging system 102. At 1006, at least one of the two or more circuit boards transmits a response to the operator console. At 1008, the operator console evaluates the responses to detect issues with the cables and/or circuit boards, as described herein and/or otherwise. At 1010, the operator console notifies the assembly line assembler that there is likely a faulty cable, a faulty cable connection and/or a faulty circuit board, as described herein and/or otherwise.

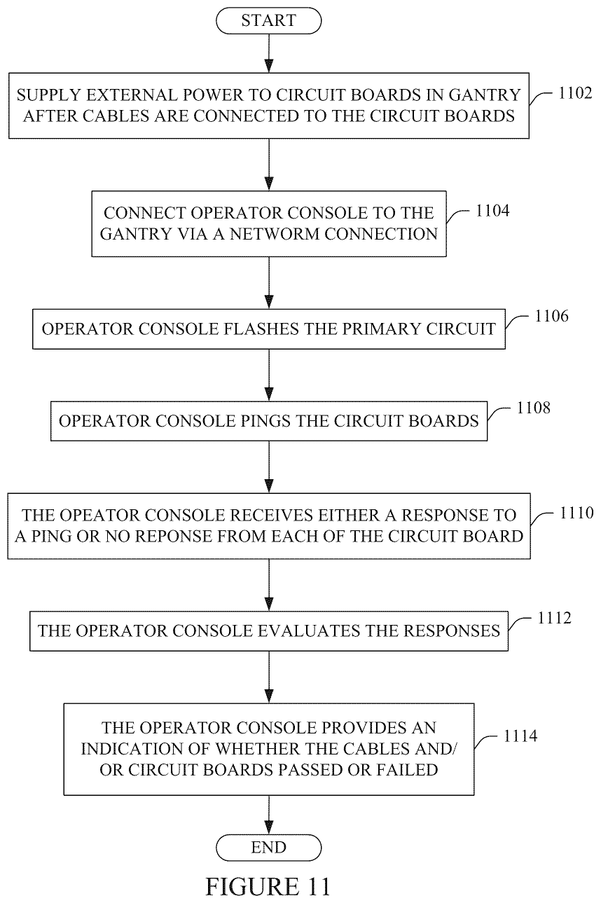

Next at FIG. 11, a non-limiting example of still another method is illustrated. It is to be appreciated that the ordering of the acts in the method is not limiting. As such, other orderings are contemplated herein. In addition, one or more acts may be omitted, and/or one or more additional acts may be included.

At 1102, external power is supplied to the circuit boards 126 after the circuit boards 126 are installed in the gantry 104 and the cables 130 are connected to the circuit boards 126, as described herein and/or otherwise. At 1104, the operator console 142 is connected to the gantry 104 via the Ethernet cable 402, as described herein and/or otherwise. At 1106, the operator console 142 flashes the primary circuit board 126ₚ, as described herein and/or otherwise.

At 1108, the operator console 142 sends ping commands to the circuit boards 126 with a timeout, as described herein and/or otherwise. At 1110, the operator console 142 either receives a response to the ping from a circuit board or receives not response (e.g., when a timeout occurs), as described herein and/or otherwise. At 1112, the operator console 142 evaluates the responses, as described herein and/or otherwise. At 1114, the operator console 142 provides an indication of whether a cable and/or a circuit boar passed or failed, as described herein and/or otherwise.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not a computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A computed tomography imaging system, comprising:
a gantry, including:
multiple circuit boards; and
a cable electrically connecting a first circuit board of the multiple circuit boards and a second circuit board of the multiple circuit boards,
wherein power is not enabled to the gantry; and
an operator console configured to transmit a test request to the first circuit board and receive responses from the first circuit board,
wherein the test request invokes the first circuit board to transmit a response request to the second circuit board, wait a predetermined time period for a response from the second circuit board, and transmit the response of the second circuit board to the operator console;
the operator console is configured to detect, based on the response, that there is a fault in at least one of the cable, a seating of the cable in a connector, and the second circuit board, and
the operator console is configured to provide a human perceivable indication of the fault in response to detecting the fault.

2. The computed tomography imaging system of claim 1, wherein the operator console supplies operator circuit board power for the multiple circuit boards.

3. The computed tomography imaging system of claim 1, wherein the gantry further includes an interface for mains power to supply operator circuit board power for the multiple circuit boards.

4. The computed tomography imaging system of claim 1, further including:
a network connection between the gantry and the operator console, wherein the test request and the response are transmitted over the network connection.

5. The computed tomography imaging system of claim 1, wherein the indication includes at least one of a visual, an audible and a haptic indication.

6. The computed tomography imaging system of claim 1, the operator console further including:
a peripheral device port, wherein the peripheral device port is in electrical connection with an external indicator device that provides the human perceivable indication.

7. The computed tomography imaging system of claim 1, wherein the first circuit board includes a memory and a processor, the operator console programs the memory with firmware prior to transmitting the test request, and the processor executes instructions of the firmware to transmit a response request to the second circuit board.

8. The computed tomography imaging system of claim 1, wherein the operator console detects the fault in response to the response of the second circuit board including no response.

9. The computed tomography imaging system of claim 1, wherein the fault includes an improper seating of the connection of the cable with at least one of the first circuit board and the second circuit board.

10. The computed tomography imaging system of claim 1, wherein the response includes an identification of the second circuit board.

11. A method, comprising:
supplying external power to circuit boards of a gantry of a CT imaging system during assembly of the CT imaging system, wherein gantry power is not enabled to the gantry during assembly of the CT imaging system;
transmitting, from an operator console of the CT imaging system, a test request to one of the circuit boards in electrical communication with one or more other circuit boards of the circuit boards via a cable,
wherein the test request invokes the one of the circuits boards to transmit a response request to the one of the other circuit boards via the cable;
receiving, at the operator console, a response of at least the one of the other circuit boards;
detecting, at the operator console and based on the response, a fault in one of a cable, a seating of a cable and the circuit boards, and
providing an indication indicating the fault is detected.

12. The method of claim 11, wherein supplying the external power includes supplying power from a power source of the operator console.

13. The method of claim 11, wherein supplying the external power includes supplying power from a mains power source.

14. The method of claim 11, further including transmitting the test request to the one of the other the circuit boards over a network connection between the operator console and the gantry.

15. The method of claim 11, wherein the indication includes at least one of a visual, an audible and a haptic indication.

16. The method of claim 11, wherein the response includes an identification of the circuit boards.

17. The method of claim 11, wherein the operator console detects the fault when the response to the response request includes no response.

18. The method of claim 11, wherein the indication includes at least one of a visual, an audible and a haptic indication.

19. The method of claim 11, wherein the indication is provided with an external indication device.

20. A system, comprising:
a first subsystem with at least multiple circuit boards and a cable electrically connecting a first circuit board of the multiple circuit boards and a second circuit board of the multiple circuit boards, wherein power is not enabled to the first subsystem;
external power for the at least multiple circuit boards; and
a second subsystem configured to communicate with the multiple circuit boards powered by the external power to detect faulty cables, faulty connections between cables and the multiple circuit boards and faulty circuit boards of the multiple circuit boards.
